(19) United States
(12) Patent Application Publication
(10) Pub. No.: US 2004/0118411 A1
(43) Pub. Date: Jun. 24, 2004

(54) Form-correcting goods for playing sports using rackets or bats or clubs

(76) Inventor: Ekapot Bhunachet, Tsukuba (JP)

Correspondence Address: Ekapot Bhunachet, 2-32-22 Kasuga, Tsukuba-shi, Ibaraki, 305-0821, JP

(21) Appl. No.: 10/324,876

(22) Filed: Dec. 21, 2002

(51) Int. Cl. A61F 5/37

(52) U.S. Cl. 128/875; 602/27; 602/19

(57) ABSTRACT

Form-correcting chest belt, forcing both arms, shoulders and chests to move in co-relation with one another, is used together with form-correcting waist belt, forcing the vertebral column to become stiff and the upper and lower parts of the body to move in co-relation with each other, and together with form-correcting shoes or foot angle supporter, which semi-force body weight center to be located in the middle of the foot length, thereby helping players play easily in the right forms the sports using rackets or bats or clubs, such as tennis, badminton, baseball and golf,

### CROSS-REFERENCE TO RELATED APPLICATIONS

National application No. JP 2002-170943 Priority date 12 Jun. 2002(12.06.02)

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

Most people, who are not professional, usually find difficulty in playing sports using rackets, bats or clubs, such as tennis, baseball or golf properly, though they have been practicing for many years. For example, in tennis, they usually hit ground stokes, both fore and back hand, with their shoulders open, i.e., twisting of the body is not enough that both shoulders can be seen from the front. With this form, players cannot control balls well and hit the balls only by hands, thus cannot hit fast and strong balls. Concerning volley, when hitting the balls with the shoulders open, ones have to pull their hands backwards beyond the shoulders. At the time of impact, players cannot see the balls because the balls and rackets are behind their heads, and of course cannot control the balls they hit.

### BRIEF DESCRIPTION OF THE INVENTION

Form-correcting goods are invented to help players of sports using rackets or bats or clubs, such as tennis, badminton, baseball and golf, play easily in the proper forms. They are composed with three parts. First is the form-correcting chest belt which force both hands, shoulders and chests to move in co-relation with one another. Second is the form-correcting waist belt which force the vertebra to become stiff, thus when twist the body the upper and lower halves will move in co-relation with each other. Third are the form-correcting shoes or foot angle supporters which semi-force the center of body weight to be in the middle of foot length when worn or fixed to the foot angle, thus help balancing the body.

### BRIEF DESCRIPTION OF THE DRAWINGS

FIG. 1 is the form-correcting chest belt observed from the front of the body.

FIG. 2 is the form-correcting chest belt observed from the back of the body.

FIG. 3 is the form-correcting waist belt observed from the back of the body.

FIG. 4 is the cut view of FIG. 3 observed from the left of the body (arrow).

FIG. 5 is the form-correcting shoe.

FIG. 6 is the form-correcting foot angle supporter.

### DETAILED DESCRIPTION OF THE INVENTION

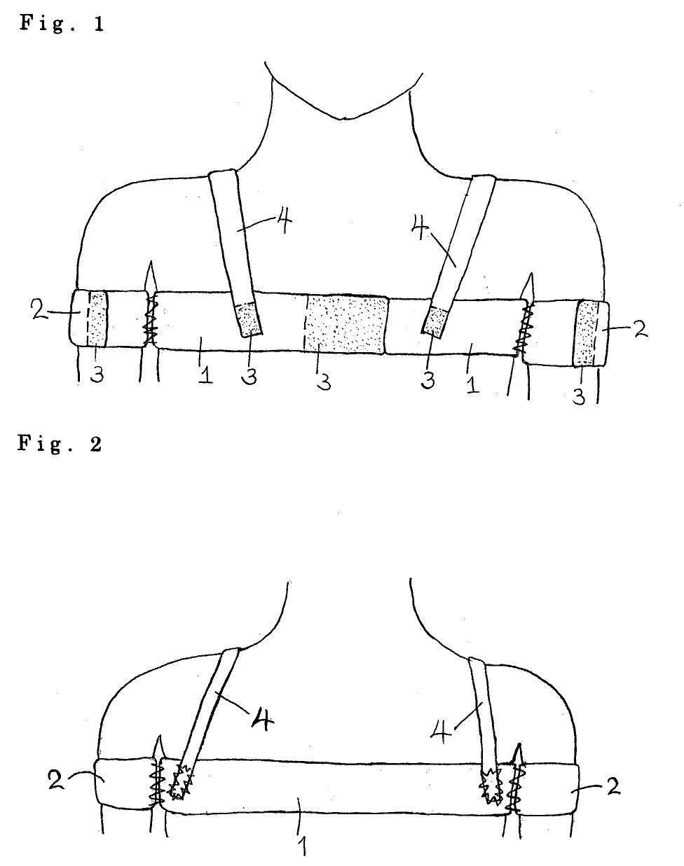

As seen in FIGS. 1 and 2, the form-correcting chest belt is composed with an elastic band wrapping around the chest (1), which is sewed to two elastic bands wrapping both upper arms (2). The ends of these elastic bands can be connected with each other using surface fastener (3). There are another two elastic bands, the shoulder bands (4), which are sewed to the chest band (1) on the back near the arms and are placed across the shoulders not crossing or crossing each other in front of/or behind the neck to be connected to the chest band using surface fastener (3). The purpose of sewing the arm bands (2) to the chest band (1) is to force the hands and chest to move together as one part. However, this purpose will be not fulfilled without the shoulder bands (4) which protect the chest band from spinning. The chest band and the shoulder bands can be replaced by an elastic fitting shirt. With the arm elastic bands sewed to this shirt, the same function as those of the form-correcting chest belt can be obtained.

Wearing this form-correcting chest belt forces both arms, shoulders and chests to move in co-relation with one another. Supposing a right-handed tennis player wearing this form-correcting chest belt is going to hit a forehand ground stoke. As his right hand draws the racket backwards, because the upper arm bases are fixed to the chest, the right shoulder and chest are forced to move together with the right arm. Simultaneously, the left chest and shoulder are also forced to turn to the right side. With this form, when looked from the front, one can only see the left shoulder. In the time of hitting the ball, the motion starts from the right chest to the right should and hand respectively, the right form, which gives a strong hit and a good ball control. The player cannot hit the ball by moving only the right arm, which is the wrong style, because the upper arm bases are fixed to the chest. In hitting the right volley, the movement is the same as hitting the forehand ground stroke, but the swing is smaller. Because the arms are forced to make an angle of about 60° with the chest, the point to hit the ball will be in front of the player. Therefore, the player can see the ball impact to the racket and make a good ball control.

In hitting the backhand ground stroke, as the player bring the racket backwards, the right chest is forced to move to the left till the right shoulder hides the left shoulder, and the motion to hit the ball starts from spinning the body by the reasons described above. The right chest serves as a base for the right hand so that the spinning force of the body can effectively transfer to the right arm and finally to the ball. This will not happen if both arms move freely not in co-relation with the chest.

After hitting the ball, good forms finish in follow through. At the moment of follow through, forearms move away from the chest. Though, until a second just before follow through, they should move together as one part. Our form-correcting chest belt, although forces both arms, shoulders and chests to move in co-relation with each other, still allows the player to do follow through, because it is made with elastic bands. Therefore, it can be used in practicing and in the real games.

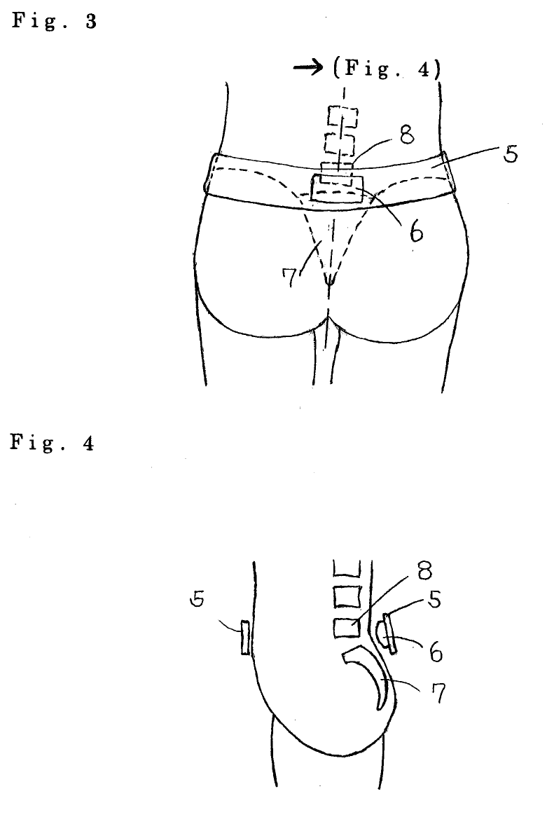

FIG. 3 shows the back view of the usage of form-correcting waist belt (5), wherein a cushion (6) is provided on the inner surface, so that, when the waist belt is put on, the cushion presses from behind the area between the sacrum (7) and the fifth lumbar vertebra (8).

FIG. 4 shows the cut view of FIG. 3 observing form the left of the body. Pressed by the cushion (6), the angle between the sacrum (7) and the fifth lumbar vertebra (8) becomes smaller, resulting in the vertebral column being stiff. The upper part and lower part of the body, therefore, move in co-relation with each other. The shape and size of the cushion (6) can be different from that written in FIGS. 3 and 4, but the place to be pressed the most has to be exactly the area between the sacrum (7) and the fifth lumbar vertebra (8). Pressing other places will not give the expected effects or even give the bad effects. The cushion can be combined with fitting wears such as girdle, waist nipper and body suit.

Using the form-correcting chest belt, together with the form-correcting waist belt, forces both arms, shoulders, chests, waists and legs to move in co-relation with each other in the correct forms. For example, a right-handed player is going to hit a forehand ground stroke. When he bring the racket backwards, the upper part of the body will move in co-relation as described. Because the vertebral column is stiff, when the right chest is moved backwards, the right thigh and leg will simultaneously turn so that the right foot becomes verticle to the ball direction. With the right foot in this position, the force of the leg pushing the ground will effectively transfer to the ball. Since, the movements of both the body upper and lower parts are in co-relation with each other, the player can quickly and sharply response to the ball.

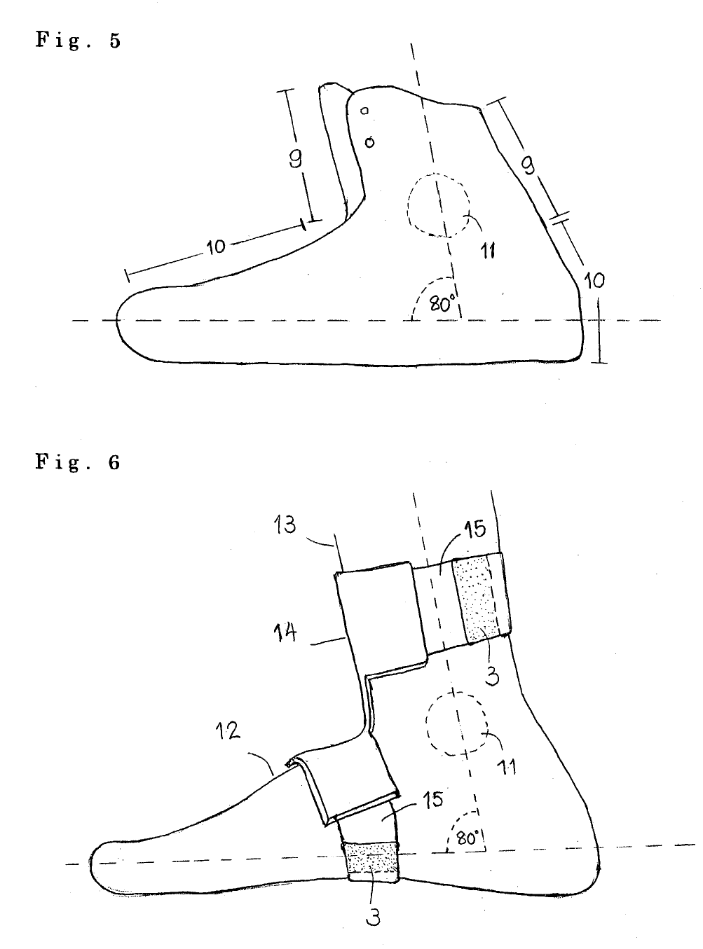

FIG. 5 shows the form-correcting shoe, which semi-forces the center of body weight to be located in the middle of the foot length when wore. Normally, when standing straight, the knee joints are fully stretched and the angle between the leg and the foot axis through the ankle is 90 degree. In this condition, the center of the body weight is not in the middle of the foot but is near to the heel. The form-correcting shoe has the foot-wrapping part (9) and the leg-wrapping part (10). When put on, the form-correcting shoe semi-forces the angle of the foot and the axis of the leg through the ankle (11) to be at about 80°. Therefore, the body weight center is semi-forced to be located in the middle of the foot length between the toe and the heel. With the body weight center in the middle of the foot length, the player can move rapidly in all directions without losing his body balance.

FIG. 6 shows the usage of the form-correcting foot angle supporter. It is composed with a supporting part (14), made of semi-elastic material such as plastic or glass fiber, and two elastic bands (15) with surface fastener (3) for fixing the supporting part to the foot (12) and leg (13). When fixed this supporter to the leg angle, the body weight center is semi-forced to be located in the middle of the foot length.

With the body weight center located in the middle of the foot length, the player can spin the body to the left or to the right and move to all direction without losing the body balance.

As described above, using the form-correcting chest belt, waist belt and shoes or foot angle supporter will help the player play tennis in the correct forms. In addition to tennis, these form-correcting goods can be used in playing other sports using racket, club or bat, such as table tennis, badminton, golf and baseball. All of these sports have the same basics that is how to transfer effectively the body spinning force through the racket or club or bat to the ball.

1. Form-correcting chest belt, for playing sports using rackets or bats or clubs, such as tennis, badminton, baseball and golf, consisting of an elastic band wrapping around the chest, which is sewed with two elastic bands wrapping both upper arms at the place just next to the shoulder joint, another two elastic bands are sewed to the chest band on the back and are placed across the shoulders to be connected with the chest bands again, so that, when the form-correcting chest belt is put on, both arms, shoulders and chests are forced to move in co-relation with one another.

2. Form-correcting waist belt, girdle, waist nipper and body suit wherein a cushion is provided on the inner surface so that, when they are put on, the cushion presses from behind the area between the fifth lumbar vertebrae and the sacrum, thereby forcing the vertebral column to become stiff and the upper and lower parts of the body to move in co-relation with each other.

3. Form-correcting shoes in which the angle between the foot-wrapping part and the leg-wrapping part is set so that when put on, semi-forces the angle between the foot and the leg axis through the ankle to be about 80°, therefore, the center of body weight is forced to locate in the middle of the foot length.

4. Form-correcting foot angle supporter, which is composed with a supporting part and two bands for fixing the support part to the foot and leg, when fixed this supporter to the leg angle the angle between the foot and the leg axis through the ankle is semi-forced to be about 80°, therefore, the center of body weight is forced to locate in the middle of the foot length.
